(19) United States
(12) Patent Application Publication
(10) Pub. No.: US 2020/0086229 A1
(43) Pub. Date: Mar. 19, 2020

(54) Method and system for shatter extraction

(71) Applicant: Nextleaf Solutions Ltd., Coquitlam (CA)

(72) Inventors: Ryan Delmoral Ko, Coquitlam (CA); Brock Hughes, Port Coquitlam (CA)

(21) Appl. No.: 16/130,193

(22) Filed: Sep. 13, 2018

(51) Int. Cl. B01D 11/02; A61K 31/352; A61K 36/185

(52) U.S. Cl. CPC B01D 11/0288; A61K 31/352; A61K 2236/50; A61K 2236/31; A61K 2236/35; A61K 36/185

(57) ABSTRACT

Concentrated cannabis extract is obtained from raw cannabis plant material using a mixed solvent extraction process. The cannabis is soaked and agitated in a chilled mixture of butane, propane and isobutane. After removing the solid plant material, the remaining solvent mixture, now with dissolved extracts, is heated to remove the solvents. The remaining, viscous oil is then heated in a vacuum oven to purge the traces of solvent and result in a brittle shatter.

### TECHNICAL FIELD

This application relates to a method and system for extracting cannabis oil concentrate from raw cannabis material. More specifically, it relates to the extraction of shatter.

### BACKGROUND

The cannabis industry is expanding in several parts of the world where it has been legalized for either recreational or medical use. For that matter, industrials are trying to create new products in order to develop their economical market. The recent legalization of cannabis and the development of this industry have led to the development of new extraction processes in order to make quality products in agreement with standards established by local governments.

Cannabis extracted from cannabis flower embodies new opportunities and safer ways to consume cannabis. Indeed, cannabis when consumed directly by smoking, introduces several components from the flower that can produce carbon chemicals, which can be harmful for the user. Therefore, smoking cannabis over a long period of time is known for its negative effects on the user. On the other hand, cannabis concentrates may represent an alternative where these negative effects can be mitigated or eliminated. During the extraction of cannabis, depending on the nature of the chemical process used, impurities such as carbohydrates, amino acids, waxes and water can be removed.

Several techniques such as solvent extraction raise new challenges especially concerning safety. In fact, the use of solvents requires more care regarding handling procedures during the process. For example, butane and propane are highly inflammable, volatile gases at room temperature and are responsible for multiple accidents every year in domestic productions. In addition to that, the extraction is run with equipment under pressure, which can raise the risk of explosion.

Among all of the smokable cannabis extracts, cannabis shatter contains a high concentration of THC (tetrahydrocannabinol) compared to other substances such as cannabis wax or oil. Cannabis shatter exhibits the advantage of being free of substances that can be potentially toxic for the user. The psychoactive effects due to the high concentration of THC are also stronger than in other ways of consumption. There is also a higher concentration of CBD (cannabidiol). Shatter has a glassy, hard and sap-like appearance.

Most of the THC in the raw cannabis plant material is contained in the part of the cannabis plant known as the trichomes. Trichomes are glass-like crystals that are capable of secreting substances such as cannabinoids and terpenoids. These molecules are mostly composed of carbon and hydrogen which make them more prone to dissolve in certain solvents. For that matter, in the solvent extraction field, the choice of solvent is important in regards of the final product consistency and purity.

This background information is provided to reveal information believed by the applicant to be of possible relevance to the present invention. No admission is necessarily intended, nor should be construed, that any of the preceding information constitutes prior art against the present invention.

### SUMMARY OF INVENTION

The present invention is directed to a method and system for extracting cannabis shatter from raw cannabis plant material via a solvent extraction process. The process is directed to extracting shatter with a high concentration of THC and a low amount of impurities. The raw cannabis plant material is soaked using a mixture of solvents, the mixture of solvents is removed from the plant material, and then the cannabis oil is separated from the solvent mixture using a heated vessel. Due to their potential toxicity, the solvents used in the extraction process have to be removed from the final extract, cannabis shatter. For this matter, the present system exhibits a purge system wherein the solvents are evaporated and reclaimed for further use.

An embodiment of the invention includes a system with a succession of vessels, pumps and cooling or heating apparatuses connected to each other through valves and hoses in such a manner as to keep the appropriate thermodynamic and process parameters under control. The success of the extraction is dependent on several factors such as the initial quality of the introduced biomass, the applied temperature and pressure, the duration of soaking, and the composition of the solvent.

Disclosed herein is a method for producing shatter from raw cannabis plant material comprising the steps of: chilling, to a temperature between −49° C. and −61° C., a mixture of butane, propane and isobutane; soaking the raw cannabis plant material in the mixture; disturbing the raw cannabis plant material during the soaking step; removing, from the raw cannabis plant material, the mixture and a cannabis oil that has dissolved in the mixture; and separating the mixture from the cannabis oil; and purging residual traces of the mixture from the cannabis oil, to result in shatter.

### BRIEF DESCRIPTION OF DRAWINGS

The following drawings illustrate embodiments of the invention and should not be construed as restricting the scope of the invention in any way.

FIG. 1 is a flowchart showing the main steps of a process to extract shatter from raw cannabis plant material, according to an embodiment of the present invention.

FIG. 2 is a flowchart describing in detail the extraction process of shatter from raw cannabis plant material, according to an embodiment of the present invention.

FIG. 3 is a schematic diagram of a system used for the extraction of shatter from raw cannabis plant material, according to an embodiment of the present invention.

### DESCRIPTION

### A. Glossary

Cannabidiol (CBD) is one of the active cannabinoids found in cannabis and is used for medicinal purposes.

The term “cannabinoids” may refer to a group of chemicals that act on cannabinoid receptors in the body, numerous of which are found in the cannabis plant.

The term “shatter” traditionally refers to butane hash oil (BHO), a cannabis extract in which butane has been used as the solvent. Herein, we use the term “shatter” irrespectively of the type of solvent used. The substance in its final form exhibits a glassy appearance and is brittle.

Tetrahydrocannabinol (THC) is a psychotropic cannabinoid and is the main psychoactive ingredient of cannabis. THC also has medicinal uses.

The term “wt” refers to a measurement by weight.

### B. Overview

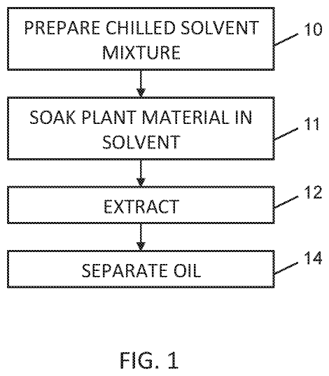

Referring to FIG. 1, shown are the main steps of an exemplary process for the extraction of shatter from raw cannabis plant material. The preparation and the chilling of the solvent mixture is performed in step 10. Specifically, the mixture of solvents includes butane, propane and isobutane, each solvent being provided separately. The solvent mixture is prepared using the ratios of 40%wt butane, 30%wt propane (instrument grade propane) and 30%wt isobutane. A 5% variance of solvent percentage is acceptable for each component of the mixture. In step 11, the raw cannabis plant material is soaked in the solvent mixture. Following this, the solvent mixture is used to extract cannabinoids from the biomass of raw cannabis plant material in step 12. Next, the cannabis oil is separated from the solvent mixture in step 14, resulting in shatter.

### C. Exemplary Embodiment

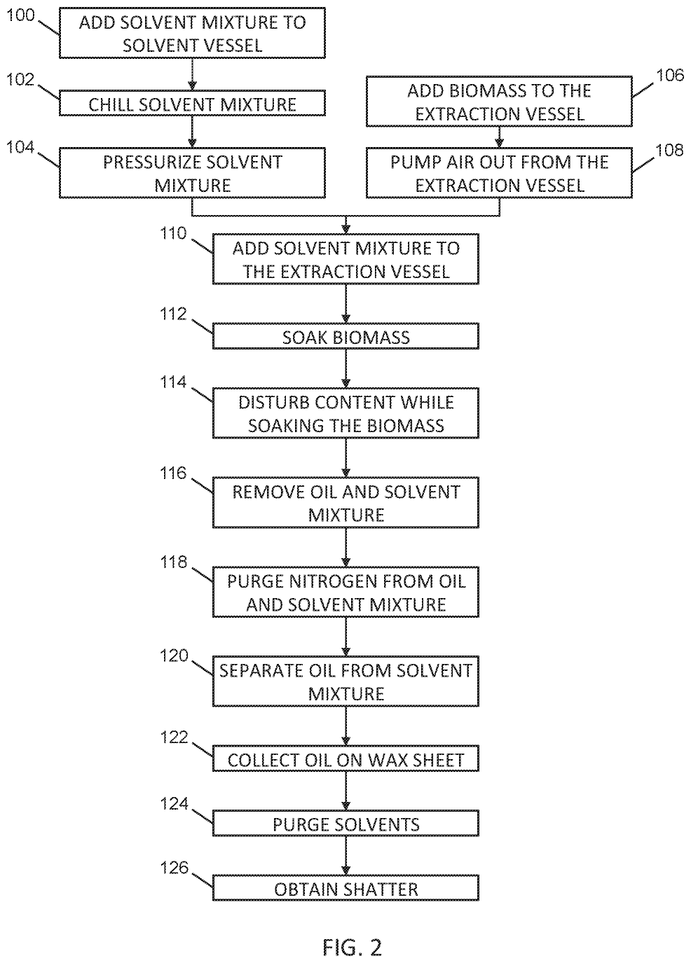

Referring to FIG. 2, there is shown an exemplary embodiment of the extraction of shatter from raw cannabis plant material in more detail. In step 100, a solvent mixture of butane, propane and isobutane in the ratio of 4:3:3 by wt % is added to the solvent vessel.

It has been found that such a composition of the solvent mixture provides for an improved quality of shatter. In step 102, the solvent is distilled into the solvent tank. The solvent vessel is chilled using CO₂, which is introduced into a jacket around the solvent tank. The solvent mixture is chilled to −55° C. in the solvent vessel by means of pressurized liquid CO₂ introduced into the jacket of the solvent vessel. Due to the chilling, the solvent mixture is kept in its liquid state. The chilling of the solvent mixture is necessary to increase the efficiency of the cannabis oil extraction. The solvent mixture in the solvent vessel is then pressurized to 40 psi (276 kPa) by introducing nitrogen gas into the solvent vessel in step 104.

In the meantime, in step 106, a biomass of raw cannabis plant material is added to the extraction vessel. The air from the extraction vessel is pumped out from the extraction vessel using a vacuum pump in step 108. Step 104 normally takes place after step 108. Step 108 is achieved prior to the transferring of the solvent mixture into the extraction vessel. A vacuum has to be created inside the extraction vessel in order to avoid a possible explosion when the pressurized solvent mixture is released inside the extraction vessel. In fact, the presence of oxygen in an environment where butane in the gaseous state is released increases the risk of explosion.

In step 110, the solvent mixture is released into the extraction vessel. This is achieved as the solvent tank is under pressure and the extraction vessel is under vacuum. In step 112, the raw cannabis plant material is soaked in the solvent mixture in the extraction vessel for half an hour. This step leads to the separation of trichomes from the raw cannabis plant materials and further to an extraction of the cannabinoids and terpenes found in the raw cannabis plant material. The trichomes are the part of the cannabis raw plant material that contain compounds such as THC, cannabinoids and terpenes. These trichomes can be found in a crystal-like state in the raw cannabis plant material.

The solvent is pushed into the extraction vessel from the solvent tank using nitrogen gas as a piston. Once the solvent is in the extraction vessel, there is no further flow of nitrogen into the extraction vessel. The extraction vessel valves are all then closed.

While the biomass is soaking in the extraction vessel, the contents of the extraction vessel are disturbed from time to time (e.g. every 5 minutes) by opening the bottom valve in order to release the pressure built up in the vessel, thereby agitating the biomass and solvent mixture, as shown in in step 114. This step helps to improve the efficiency of the soaking process by promoting the increase of the exposed surface area between the biomass and solvent mixture and enhancing the extraction process.

Then, in step 116, the solvent mixture now with dissolved cannabis oil is removed from the extraction vessel and transferred to a separation vessel in order to separate the cannabis oil from the solvent mixture. Some of the nitrogen passes into the separation vessel along with solvent and crude cannabis oil. In order to reduce the pressure in the separation vessel, the nitrogen is purged out from the separation vessel in step 118, before separating the solvent from the cannabis oil. In step 120, the separation vessel is heated with warm water or another heating media at 30-32° C. for triggering the evaporation and the collection of the solvent mixture for further use. This reclamation of solvent is monitored by keeping track of pressure in the separator vessel. When the solvent has all been removed, the pressure in the vessel decreases completely. The applied temperature is sufficient to trigger the transition of the solvents to the gaseous state.

After that, the resulting, viscous cannabis oil is collected on a parchment paper sheet in step 122. The parchment paper along with poured cannabis oil is transferred into a vacuum oven at a temperature between 30-90° C. and kept there for 3-4 days to remove traces of solvent.

The solvent is removed, in purge step 124, by heating it under vacuum for 3-4 day to remove traces of solvent from the cannabis oil concentrate. Finally, the shatter is obtained in step 126. The shatter may then be stored preferentially in an airtight and light-proof container in order to prevent degradation. The storage of shatter in a cool environment is also a requirement for keeping the product in good condition for an extended period of time.

### D. Apparatus

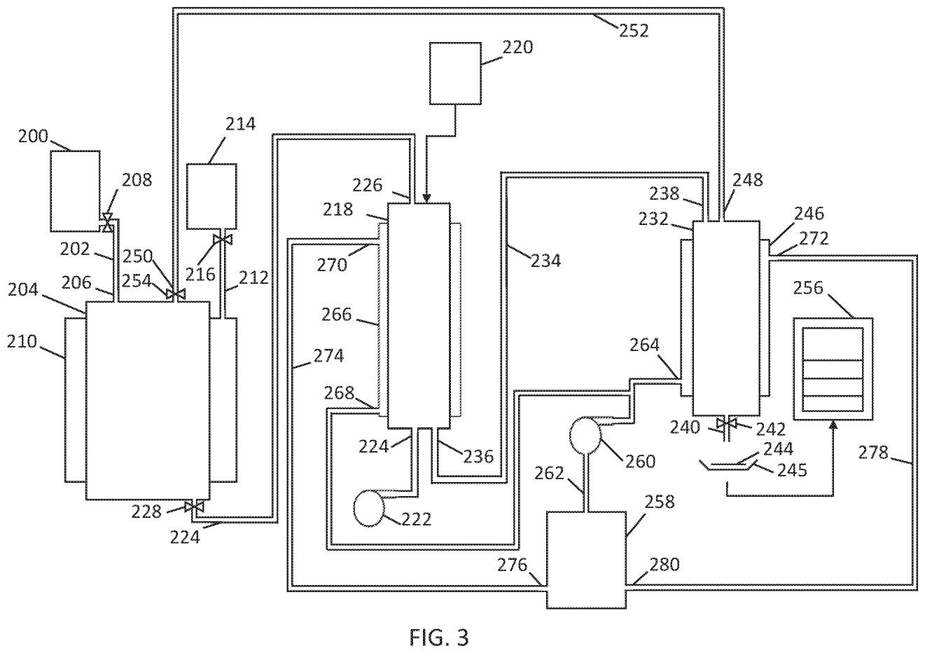

Referring to FIG. 3, a schematic diagram shows an embodiment of the shatter extraction system. A pressurized nitrogen cylinder 200 is connected via a hose 202 to a solvent vessel 204. The hose 202 is connected to the inlet 206 of the solvent vessel 204. The solvent vessel 204 may be a stainless-steel canister, for example. A ball valve 208 is mounted on the nitrogen cylinder 200 in order to release the nitrogen gas into the solvent vessel 204 when needed. The solvent mixture is, in one embodiment, a blend of butane (40 wt %), propane (30 wt %) and isobutane (30 wt %).

The solvent vessel 204 has a jacket 210 connected via a hose 212 to a liquefied CO₂ cylinder 214. The CO₂ contained in CO₂ cylinder 214 is released into the jacket 210 with the help of a ball valve 216 mounted on the CO₂ cylinder. The pressure inside the CO₂ cylinder 214 is in the range of 2-2.4 MPa (300-350 psi). The desired temperature in the solvent vessel 204 is −55° C. with a pressure of 276 kPa (40 psi). The CO₂ in the jacket 210 of the solvent vessel 204 acts as a coolant for the solvent mixture. Chilling the solvent helps to eliminate the impurities in the extracted crude oil. The pressure and the temperature inside the solvent vessel 204 may be measured with the help of gauges mounted on one of the ball valves. In some embodiments, the gauges are directly set up inside the solvent vessel 204, or inserted into the sidewall or top of the solvent vessel.

Before releasing the solvent mixture from the solvent vessel 204 into the extraction vessel 218, the biomass of raw cannabis plant material 220 is added to the extraction vessel 218. Approximately, between 1-1.5 kg (2.5-3 pounds) of raw cannabis plant material is introduced into the extraction vessel 218. Then, a vacuum is created inside the extraction vessel 218 containing the raw cannabis plant material using a vacuum pump 222 connected to the extraction vessel via an outlet 224.

When the extraction vessel 218 is ready for the soaking process i.e. the extraction vessel containing the raw cannabis plant material is under vacuum, the solvent mixture is released from the solvent vessel 204 to the extraction vessel. The solvent vessel 204 is connected via a hose 224 connected to an inlet 226 of the extraction vessel 218. The release of the solvent mixture from the solvent vessel 204 is triggered by opening the ball valve 228 mounted on the solvent vessel.

During the soaking process, the extraction vessel 218 is disturbed at an interval of 5 minutes by opening and closing the drain valve 236. The drain valve 236 is opened and closed quickly, by just enough to disrupt/shake the mixture of solvent and biomass in the extraction vessel. The aim of this operation is to induce agitation of the contents of the extraction vessel in order to improve the efficiency of the soaking process, by increasing the contact surface area between the raw plant cannabis material and the solvent mixture.

After 30 minutes, the solvent mixture with dissolved cannabis oil is transferred to the separation vessel 232 through a hose 234 connected from the outlet 236 of the extraction vessel 218 to the inlet 238 of the separation vessel 232. In the separation vessel 232 the cannabis oil is separated from the solvent mixture and collected, via an outlet 240 with a ball valve 242, on a clean wax sheet 244 on a tray 245. The separation vessel 232 is heated by a jacket 246 filled with hot water at 30-32° C. This process is applied in order to evaporate the solvent mixture, which is composed of solvents that have low boiling points.

The solvent mixture is then collected during the process from an outlet 248 of the separation vessel 232 connected to the inlet 250 of the solvent vessel 204 through a hose 252. A ball valve 254 is mounted on the inlet 250 of the solvent vessel 204. This closed loop setup for the solvent mixture exhibits the advantage of reducing the potential risk of explosion since the solvent is not released into the atmosphere in the vicinity of the apparatus.

The shatter is then placed in a vacuum oven 256 for a 3-4 day heating treatment.

A hot water reservoir 258 containing water at 30-32° C. is connected to a pump 260 via a hose 262. The hot water is pumped into the jacket 246 of the separation vessel 232 via the inlet 264 and into the jacket 266 of the extraction vessel 218 via the inlet 268. The extraction vessel is heated to reclaim any residual or trapped solvents from the biomass. The hot water is reclaimed from the jacket 266 of the extraction vessel 218 via the outlet 270 and from the jacket 246 of the separation vessel 232 via the outlet 272. The outlet 270 of the extraction vessel 218 is connected through a hose 274 to an inlet 276 located on the hot water reservoir 258. The hot water is reclaimed from the jacket 246 of the separation vessel 232 via the outlet 272 through a hose 278 connected to the inlet 280 of the hot water reservoir 258

### E. Variations

While the present embodiment is of the best presently contemplated mode of carrying out the subject matter disclosed and claimed herein, other embodiments are possible.

In various embodiments, a different ratio of solvents in the solvent mixture can be used for extracting the cannabis oil from the raw plant material. For example, the butane proportion can be in the range 45-55 wt %, the propane proportion can be in the range 27-33 wt %, and isobutane can make up the remainder of the solvent mixture.

In some embodiments, other vessels can be mounted in parallel or in series in the loop. In some embodiments, a different cooling system can be applied to the various vessels. In some embodiments, a different heating system can be applied to the various vessels.

In other embodiments within the purview of the present invention, other plant materials besides cannabis may be processed.

Temperatures that have been given to the nearest degree include all temperatures within a range of ±0.5° C. of the given value. Quantities may be different in other embodiments.

It is expected that the process will work adequately if the temperature to which the solvent mixture is chilled is within 10% of −55° C., i.e. from −61° C. to −49° C. Similarly, it is expected that the process will work adequately if the hot water is at a temperature within a higher range, e.g. anywhere from 29° C. to 33° C. Other soaking duration may be used, for example 20-40 minutes.

In other embodiments, the solvent mixture in the solvent vessel is pressurized to 35-40 psi (241-276 kPa) by introducing nitrogen gas into the solvent vessel.

In general, unless otherwise indicated, singular elements may be in the plural and vice versa with no loss of generality.

Throughout the description, specific details have been set forth in order to provide a more thorough understanding of the invention. However, the invention may be practiced without these particulars. In other instances, well known elements have not been shown or described in detail and repetitions of steps and features have been omitted to avoid unnecessarily obscuring the invention. For example, various pumps, valves, jackets and lines are not shown for clarity. Accordingly, the specification and drawings are to be regarded in an illustrative, rather than a restrictive, sense.

It will be clear to one having skill in the art that further variations to the specific details disclosed herein can be made, resulting in other embodiments that are within the scope of the invention disclosed. Steps in the flowchart may be performed in a different order, other steps may be added, or one or more may be removed without altering the main outcome of the process. Various parameters and configurations described with respect to a specific embodiment are examples only and may be changed depending on the specific embodiment. Accordingly, the scope of the invention is to be construed in accordance with the substance defined by the following claims.

1. A method for producing shatter from raw cannabis plant material comprising the steps of:
chilling, to a temperature between −49° C. and −61° C., a mixture of butane, propane and isobutane;
soaking the raw cannabis plant material in the mixture;
disturbing the raw cannabis plant material during the soaking step;
removing, from the raw cannabis plant material, the mixture and a cannabis oil that has dissolved in the mixture; and
separating the mixture from the cannabis oil; and
purging residual traces of the mixture from the cannabis oil, to result in shatter.

2. The method of claim 1, wherein said mixture has butane, propane and isobutane in a ratio of 4:3:3.

3. The method of claim 1, wherein a nitrogen gas is used to push the mixture from a solvent tank to an extraction vessel.

4. The method of claim 1, wherein the soaking step lasts for 30 minutes.

5. The method of claim 1, wherein the raw cannabis plant material is disturbed every 5 minutes during the soaking step.

6. The method of claim 1, wherein the separating step is carried out at a temperature between 30-32° C.

7. The method of claim 1, wherein the mixture is chilled to −55° C.

8. The method of claim 1, further comprising collecting the cannabis oil on parchment paper prior to placing it in a vacuum oven.

9. The method of claim 1, wherein the traces of mixture are removed by heating under vacuum for 3-4 days at a temperature between 29-34° C.

10. The method of claim 1 wherein:
the chilling occurs in a solvent vessel;
the soaking occurs in an extraction vessel;
the separating occurs in a separation vessel; and
the purging occurs in a vacuum oven.

11. The method of claim 10, further comprising introducing liquefied CO₂ into a jacket around the solvent vessel in order to chill the mixture.

12. The method of claim 10, further comprising pressurizing the solvent vessel with nitrogen gas to a pressure of 138-414 kPa.

13. The method of claim 10, further comprising introducing water at 30-32° C. into a jacket around the extraction vessel and a jacket around the separation vessel.

14. The method of claim 10, further comprising opening an outlet valve mounted on the extraction vessel to disturb the raw cannabis plant material while soaking.

15. The method of claim 14, wherein the outlet valve is opened and closed intermittently during the soaking step.

16. The method of claim 10, further comprising transferring the mixture from the separation vessel directly to the solvent vessel during the separation step, thereby reclaiming the mixture.

17. The method of claim 10, further comprising:
placing the raw cannabis plant material in the extraction vessel;
evacuating air from the extraction vessel; and
adding the mixture to the evacuated solvent vessel.
